(19) United States
(12) Patent Application Publication
(10) Pub. No.: US 2009/0312591 A1
(43) Pub. Date: Dec. 17, 2009

(54) METHOD FOR PRODUCING PROPENE FROM PROPANE

(75) Inventors: Markus Schubert, Ludwigshafen (DE); Ulrich Müller, Neustadt (DE); Christoph Kiener, Weisenheim Am Sand (DE); Friedhalm Teich, Neckarhausen (DE); Sven Crone, Limburgerhof (DE); Falk Simon, Bensheim (DE); Jörg Pastre, Bensheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/294,748

(22) PCT Filed: Mar. 13, 2007
(86) PCT No.: PCT/EP2007/052354

§ 371 Date: Sep. 26, 2008

(30) Foreign Application Priority Data
Mar. 29, 2006 (EP) 06111922.8

(51) Int. Cl. C07C 5/327

(52) U.S. Cl. 585/654

(57) ABSTRACT

The present invention relates to processes for preparing propene from propane, comprising the steps:
A) a feed gas stream a comprising propane is provided;
B) the feed gas stream a comprising propane, if appropriate steam and if appropriate and an oxygenous gas stream are fed into a dehydrogenation zone and propane is subjected to a dehydrogenation to propene to obtain a product gas stream b comprising propane, propene, methane, ethane, ethene, hydrogen, if appropriate carbon monoxide, carbon dioxide, steam and oxygen;
C) the product gas stream b is cooled, if appropriate compressed and steam is removed by condensation to obtain a steam-depleted product gas stream c;
D) the product gas stream c is contacted with a selective adsorbent which adsorbs propene selectively under the selected adsorption conditions to obtain a propene-laden adsorbent and a propene-depleted gas stream d2 comprising propane, methane, ethane, ethene and hydrogen, carbon monoxide and carbon dioxide;
E) a propene-comprising gas stream e1 is released from the propane-laden adsorbent by pressure reduction and/or heating of the adsorbent.

The invention relates to a process for preparing propene from propane.

Propene is obtained on the industrial scale by dehydrogenating propane.

In the process, known as the UOP-oleflex process, for dehydrogenating propane to propene, a feed gas stream comprising propane is preheated to 600-700° C. and dehydrogenated in a moving bed dehydrogenation reactor over a catalyst which comprises platinum on alumina to obtain a product gas stream comprising predominantly propane, propene and hydrogen. In addition, low-boiling hydrocarbons formed by cracking (methane, ethane, ethene) and small amounts of high boilers (C₄⁺ hydrocarbons) are present in the product gas stream. The product gas mixture is cooled and compressed in a plurality of stages. Subsequently, the C₂ and C₃ hydrocarbons and the high boilers are removed from the hydrogen and methane formed in the dehydrogenation by condensation in a “cold box”. The liquid hydrocarbon condensate is subsequently separated by distillation by removing the C₂ hydrocarbons and remaining methane in a first column and separating the C₃ hydrocarbon stream into a propene fraction having high purity and a propane fraction which also comprises the C₄⁺ hydrocarbons in a second distillation column.

A disadvantage of this process is the loss of C₃ hydrocarbons by the condensation in the cold box. Owing to the large amounts of hydrogen formed in the dehydrogenation and as a consequence of the phase equilibrium, relatively large amounts of C₃ hydrocarbons are also discharged with the hydrogen/methane offgas stream unless condensation is effected at very low temperatures. Thus, it is necessary to work at temperatures of from −20 to -120° C. in order to limit the loss of C₃ hydrocarbons which are discharged with the hydrogen/methane offgas stream.

It is an object of the present invention to provide an improved process for dehydrogenating propane to propene.

The object is achieved by a process for preparing propene from propane, comprising the steps:
A) a feed gas stream a comprising propane is provided;
B) the feed gas stream a comprising propane, if appropriate steam and if appropriate an oxygenous gas stream are fed into a dehydrogenation zone and propane is subjected to a dehydrogenation to propene to obtain a product gas stream b comprising propane, propene, methane, ethane, ethene, hydrogen, if appropriate carbon monoxide, carbon dioxide, steam and oxygen;
C) the product gas stream b is cooled, if appropriate compressed and steam is removed by condensation to obtain a steam-depleted product gas stream c;
D) the product gas stream c is contacted with a selective adsorbent which adsorbs propene selectively to obtain a propene-laden adsorbent and a propene-depleted gas stream d2 comprising propane, methane, ethane, ethene and hydrogen, if appropriate carbon monoxide and carbon dioxide;
E) a propene-comprising gas stream e1 is released from the propane-laden adsorbent by pressure reduction and/or heating of the adsorbent.

In a first process part, A), a feed gas stream a comprising propane is provided. This generally comprises at least 80% by volume of propane, preferably 90% by volume of propane. In addition, the propane-containing feed gas stream a generally also comprises butanes (n-butane, isobutane). Typical compositions of the propane-containing feed gas stream are disclosed in DE-A 102 46 119 and DE-A 102 45 585. Typically, the propane-containing feed gas stream a is obtained from liquid petroleum gas (LPG).

In one process part, B), the feed gas stream comprising propane is fed into a dehydrogenation zone and subjected to a generally catalytic dehydrogenation. In this process part, propane is dehydrogenated partially in a dehydrogenation reactor over a dehydrogenation-active catalyst to give propene. In addition, hydrogen and small amounts of methane, ethane, ethene and C₄⁺ hydrocarbons (n-butane, isobutane, butenes, butadiene) are obtained. Also generally obtained in the product gas mixture of the catalytic propane dehydrogenation are carbon oxides (CO, CO₂), in particular CO₂, steam and if appropriate inert gases to a small degree. The product gas stream of the dehydrogenation generally comprises steam which has already been added to the dehydrogenation gas mixture and/or, in the case of dehydrogenation in the presence of oxygen (oxidative or non-oxidative), has already formed in the dehydrogenation. When the dehydrogenation is carried out in the presence of oxygen, inert gases (nitrogen) are introduced into the dehydrogenation zone with the oxygen-containing gas stream fed in, as long as pure oxygen is not fed in. When an oxygenous gas is fed in, its oxygen content is generally at least 40% by volume, preferably at least 80% by volume, more preferably at least 90% by volume. Especially technically pure oxygen with an oxygen content of >99% is employed, in order to avoid too high an inert gas fraction in the product gas mixture. In addition, unconverted propane is present in the product gas mixture.

The propane dehydrogenation can in principle be carried out in any reactor types known from the prior art. A comparatively comprehensive description of reactor types suitable in accordance with the invention is contained in “Catalytica® Studies Division, Oxidative Dehydrogenation and Alternative Dehydrogenation Processes” (Study Number 4192 OD, 1993, 430 Ferguson Drive, Mountain View, Calif., 94043-5272, USA).

The dehydrogenation can be carried out as an oxidative or non-oxidative dehydrogenation. The dehydrogenation can be carried out isothermally or adiabatically. The dehydrogenation can be carried out catalytically in a fixed bed, moving bed or fluidized bed reactor.

The nonoxidative catalytic propane dehydrogenation is preferably carried out autothermally. To this end, oxygen is additionally admixed with the reaction gas mixture of the propane dehydrogenation in at least one reaction zone and the hydrogen and/or hydrocarbon present in the reaction gas mixture is at least partly combusted, which directly generates in the reaction gas mixture at least some of the heat required for dehydrogenation in the at least one reaction zone.

One feature of the nonoxidative method compared to an oxidative method is the formation of hydrogen at least as an intermediate, which is manifested in the presence of hydrogen in the product gas of the dehydrogenation. In the oxidative dehydrogenation, there is no free hydrogen in the product gas of the dehydrogenation.

A suitable reactor form is the fixed bed tubular or tube bundle reactor. In these reactors, the catalyst (dehydrogenation catalyst and if appropriate a specialized oxidation catalyst) is disposed as a fixed bed in a reaction tube or in a bundle of reaction tubes. Customary reaction tube internal diameters are from about 10 to 15 cm. A typical dehydrogenation tube bundle reactor comprises from about 300 to 1000 reaction tubes. The internal temperature in the reaction tubes typically varies in the range from 300 to 1200° C., preferably in the range from 500 to 1000° C. The working pressure is customarily from 0.5 to 8 bar, frequently from 1 to 2 bar, when a low steam dilution is used, or else from 3 to 8 bar when a high steam dilution is used (corresponding to the steam active reforming process (STAR process) or the Linde process) for the dehydrogenation of propane or butane of Phillips Petroleum Co. Typical gas hourly space velocities (GHSV) are from 500 to 2000 h⁻¹, based on hydrocarbon used. The catalyst geometry may, for example, be spherical or cylindrical (hollow or solid).

The catalytic propane dehydrogenation may also be carried out under heterogeneous catalysis in a fluidized bed, according to the Snamprogetti/Yarsintez-FBD process. Appropriately, two fluidized beds are operated in parallel, of which one is generally in the state of regeneration.

The working pressure is typically from 1 to 2 bar, the dehydrogenation temperature generally from 550 to 600° C. The heat required for the dehydrogenation can be introduced into the reaction system by preheating the dehydrogenation catalyst to the reaction temperature. The admixing of a cofeed comprising oxygen at least allows the preheater to be dispensed with partly and the required heat to be generated directly in the reactor system by combustion of hydrogen and/or hydrocarbons in the presence of oxygen. If appropriate, a cofeed comprising hydrogen may additionally be admixed.

The catalytic propane dehydrogenation can be carried out in a tray reactor. When the dehydrogenation is carried out autothermally with an oxygenous gas stream being fed in, it is preferably carried out in a tray reactor. This reactor comprises one or more successive catalyst beds. The number of catalyst beds may be from 1 to 20, advantageously from 1 to 6, preferably from 1 to 4 and in particular from 1 to 3. The catalyst beds are preferably flowed through radially or axially by the reaction gas. In general, such a tray reactor is operated using a fixed catalyst bed. In the simplest case, the fixed catalyst beds are disposed axially in a shaft furnace reactor or in the annular gaps of concentric cylindrical grids. A shaft furnace reactor corresponds to one tray. The performance of the dehydrogenation in a single shaft furnace reactor corresponds to one embodiment. In a further, preferred embodiment, the dehydrogenation is carried out in a tray reactor having 3 catalyst beds.

In general, the amount of the oxygenous gas added to the reaction gas mixture is selected in such a way that the amount of heat required for the dehydrogenation of the propane is generated by the combustion of the hydrogen present in the reaction gas mixture and of any hydrocarbons present in the reaction gas mixture and/or of carbon present in the form of coke. In general, the total amount of oxygen supplied, based on the total amount of propane, is from 0.001 to 0.8 mol/mol, preferably from 0.001 to 0.6 mol/mol, more preferably from 0.02 to 0.5 mol/mol. Oxygen may be used either in the form of pure oxygen or in the form of oxygenous gas which comprises inert gases. In order to avoid high propane and propene losses in the workup (see below), it is, however, essential that the oxygen content of the oxygenous gas used is high and is at least 40% by volume, preferably at least 80% by volume, more preferably at least 90% by volume. Particularly preferred oxygenous gas is technically pure oxygen having an O₂ content of approx. 99% by volume.

The hydrogen combusted to generate heat is the hydrogen formed in the catalytic propane dehydrogenation and also any hydrogen additionally added to the reaction gas mixture as hydrogenous gas. The amount of hydrogen present should preferably be such that the molar H₂/O₂ ratio in the reaction gas mixture immediately after oxygen is fed in is from 1 to 10 mol/mol, preferably from 2 to 5 mol/mol. In multistage reactors, this applies to every intermediate feed of oxygenous and any hydrogenous gas.

The hydrogen is combusted catalytically. The dehydrogenation catalyst used generally also catalyzes the combustion of the hydrocarbons and of hydrogen with oxygen, so that in principle no specialized oxidation catalyst is required apart from it. In one embodiment, operation is effected in the presence of one or more oxidation catalysts which selectively catalyze the combustion of hydrogen with oxygen in the presence of hydrocarbons. The combustion of these hydrocarbons with oxygen to give CO, CO₂ and water therefore proceeds only to a minor extent. The dehydrogenation catalyst and the oxidation catalyst are preferably present in different reaction zones.

When the reaction is carried out in more than one stage, the oxidation catalyst may be present only in one, in more than one or in all reaction zones.

Preference is given to disposing the catalyst which selectively catalyzes the oxidation of hydrogen at the points where there are higher partial oxygen pressures than at other points in the reactor, in particular near the feed point for the oxygenous gas. The oxygenous gas and/or hydrogenous gas may be fed in at one or more points in the reactor.

In one embodiment of the process according to the invention, there is intermediate feeding of oxygenous gas and of hydrogenous gas upstream of each tray of a tray reactor. In a further embodiment of the process according to the invention, oxygenous gas and hydrogenous gas are fed in upstream of each tray except the first tray. In one embodiment, a layer of a specialized oxidation catalyst is present downstream of every feed point, followed by a layer of the dehydrogenation catalyst. In a further embodiment, no specialized oxidation catalyst is present. The dehydrogenation temperature is generally from 400 to 1100° C.; the pressure in the last catalyst bed of the tray reactor is generally from 0.2 to 15 bar, preferably from 1 to 10 bar, more preferably from 1 to 5 bar. The GHSV is generally from 500 to 2000 h⁻¹, and, in high-load operation, even up to 100 000 h⁻¹, preferably from 4000 to 16 000 h⁻¹.

A preferred catalyst which selectively catalyzes the combustion of hydrogen comprises oxides and/or phosphates selected from the group consisting of the oxides and/or phosphates of germanium, tin, lead, arsenic, antimony and bismuth. A further preferred catalyst which catalyzes the combustion of hydrogen comprises a noble metal of transition group VIII and/or I of the periodic table.

The dehydrogenation catalysts used generally comprise a support and an active composition. The support generally consists of a heat-resistant oxide or mixed oxide. The dehydrogenation catalysts preferably comprise a metal oxide which is selected from the group consisting of zirconium dioxide, zinc oxide, aluminum oxide, silicon dioxide, titanium dioxide, magnesium oxide, lanthanum oxide, cerium oxide and mixtures thereof, as a support. The mixtures may be physical mixtures or else chemical mixed phases such as magnesium aluminum oxide or zinc aluminum oxide mixed oxides. Preferred supports are zirconium dioxide and/or silicon dioxide, and particular preference is given to mixtures of zirconium dioxide and silicon dioxide.

Suitable shaped catalyst body geometries are extrudates, stars, rings, saddles, spheres, foams and monoliths with characteristic dimensions of from 1 to 100 mm.

The active composition of the dehydrogenation catalysts generally comprises one or more elements of transition group VIII of the periodic table, preferably platinum and/or palladium, more preferably platinum. Furthermore, the dehydrogenation catalysts may comprise one or more elements of main group I and/or II of the periodic table, preferably potassium and/or cesium. The dehydrogenation catalysts may further comprise one or more elements of transition group III of the periodic table including the lanthanides and actinides, preferably lanthanum and/or cerium. Finally, the dehydrogenation catalysts may comprise one or more elements of main group III and/or IV of the periodic table, preferably one or more elements from the group consisting of boron, gallium, silicon, germanium, tin and lead, more preferably tin.

In a preferred embodiment, the dehydrogenation catalyst comprises at least one element of transition group VIII, at least one element of main group I and/or II, at least one element of main group III and/or IV and at least one element of transition group III including the lanthanides and actinides.

For example, all dehydrogenation catalysts which are disclosed by WO 99/46039, U.S. Pat. No. 4,788,371, EP-A 705 136, WO 99/29420, U.S. Pat. No. 5,220,091, U.S. Pat. No. 5,430,220, U.S. Pat No. 5,877,369, EP 0 117 146, DE-A 199 37 106, DE-A 199 37 105 and DE-A 199 37 107 may be used in accordance with the invention. Particularly preferred catalysts for the above-described variants of autothermal propane dehydrogenation are the catalysts according to examples 1, 2, 3 and 4 of DE-A 199 37 107.

Preference is given to carrying out the autothermal propane dehydrogenation in the presence of steam. The added steam serves as a heat carrier and supports the gasification of organic deposits on the catalysts, which counteracts carbonization of the catalysts and increases the onstream time of the catalysts. This converts the organic deposits to carbon monoxide, carbon dioxide and if appropriate water. The dilution with steam shifts the equilibrium conversion of the dehydrogenation.

The dehydrogenation catalyst may be regenerated in a manner known per se. For instance, steam may be added to the reaction gas mixture or a gas comprising oxygen may be passed from time to time over the catalyst bed at elevated temperature and the deposited carbon burnt off. After the regeneration, the catalyst is reduced with a hydrogenous gas if appropriate.

The product gas stream b may be separated into two substreams, in which case one substream is recycled into the autothermal dehydrogenation, corresponding to the cycle gas method described in DE-A 102 11 275 and DE-A 100 28 582.

The propane dehydrogenation may be carried out as an oxidative dehydrogenation. The oxidative propane dehydrogenation may be carried out as a homogeneous oxidative dehydrogenation or as a heterogeneously catalyzed oxidative dehydrogenation.

When the propane dehydrogenation in the process according to the invention is configured as a homogeneous oxydehydrogenation, this can in principle be carried out as described in the documents U.S. Pat. No. 3,798,283, CN-A 1,105,352, Applied Catalysis, 70 (2), 1991, p. 175 to 187, Catalysis Today 13, 1992, p. 673 to 678 and the DE-A 1 96 22 331.

The temperature of the homogeneous oxydehydrogenation is generally from 300 to 700° C., preferably from 400 to 600° C., more preferably from 400 to 500° C. The pressure may be from 0.5 to 100 bar or from 1 to 50 bar. It will frequently be from 1 to 20 bar, in particular from 1 to 10 bar.

The residence time of the reaction gas mixture under oxydehydrogenation conditions is typically from 0.1 or 0.5 to 20 sec, preferably from 0.1 or 0.5 to 5 sec. The reactor used may, for example, be a tube oven or a tube bundle reactor such as a countercurrent tube oven with flue gas as a heat carrier, or a tube bundle reactor with salt melt as a heat carrier.

The propane to oxygen ratio in the starting mixture to be used may be from 0.5:1 to 40:1. The molar ratio of propane to molecular oxygen in the starting mixture is preferably ≦6:1, more preferably ≦5:1. In general, the aforementioned ratio will be ≧1:1, for example ≧2:1. The starting mixture may comprise further, substantially inert constituents such as H₂O, CO₂, CO, N₂, noble gases and/or propene. Propene may be present in the C₃ fraction coming from the refinery. It is favorable for a homogeneous oxidative dehydrogenation of propane to propene when the ratio of the surface area of the reaction space to the volume of the reaction space is at a minimum, since the homogeneous oxidative propane dehydrogenation proceeds by a free-radical mechanism and the reaction chamber surface generally functions as a free-radical scavenger. Particularly favorable surface materials are aluminas, quartz glass, borosilicates, stainless steel and aluminum.

When the first reaction stage in the process according to the invention is configured as a heterogeneously catalyzed oxydehydrogenation, this can in principle be carried out as described in the documents U.S. Pat. No. 4,788,371, CN-A 1,073,893 Catalysis Letters 23 (1994) 103-106, W. Zhang, Gaodeng Xuexiao Huaxue Xuebao, 14 (1993) 566, Z. Huang, Shiyou Huagong, 21 (1992) 592, WO 97/36849, DE-A 1 97 53 817, U.S. Pat. No. 3,862,256, U.S. Pat. No. 3,887,631, DE-A 1 95 30 454, U.S. Pat. No. 4,341,664, J. of Catalysis 167, 560-569 (1997), J. of Catalysis 167, 550-559 (1997), Topics in Catalysis 3 (1996) 265-275, U.S. Pat. No. 5,086,032, Catalysis Letters 10 (1991) 181-192, Ind. Eng. Chem. Res. 1996, 35, 14-18, U.S. Pat. No. 4,255,284, Applied Catalysis A: General, 100 (1993) 111-130, J. of Catalysis 148, 56-67 (1994), V. Cortés Corberán and S. Vic Bellón (Editors), New Developments in Selective Oxidation II, 1994, Elsevier Science B.V., p. 305-313, 3rd World Congress on Oxidation Catalysis R. K. Grasselli, S. T. Oyama, A. M. Gaffney and J. E. Lyons (Editors), 1997, Elsevier Science B.V., p. 375 ff. In particular, all of the oxydehydrogenation catalysts specified in the aforementioned documents may be used. The statement made for the abovementioned documents also applies to:

i) Otsuka, K.; Uragami, Y.; Komatsu, T.; Hatano, M. in Natural Gas Conversion, Stud. Surf. Sci. Catal.; Holmen A.; Jens, K.-J.; Kolboe, S., Eds.; Elsevier Science: Amsterdam, 1991; Vol. 61, p 15;
ii) Seshan, K.; Swaan, H. M.; Smits, R. H. H.; van Ommen, J. G.; Ross, J. R. H. in New Developments in Selective Oxidation; Stud. Surf. Sci. Catal.; Centi, G.; Trifird, F., Eds; Elsevier Science: Amsterdam 1990; Vol. 55, p 505;
iii) Smits, R. H. H.; Seshan, K.; Ross, J. R. H. in New Developments in Selective Oxidation by Heterogeneous Catalysis; Stud. Surf. Sci. Catal; Ruiz, P.; Delmon, B., Eds.; Elsevier Science: Amsterdam, 1992 a; Vol. 72, p 221;

### iv) Smits, R. H. H.; Seshan, K.; Ross, J. R. H. Proceedings, Symposium on Catalytic Selective Oxidation, Washington D.C.; American Chemical Society: Washington, D.C., 1992 b; 1121;

v) Mazzocchia, C.; Aboumrad, C.; Daigne, C.; Tempesti, E.; Herrmann, J. M.; Thomas, G. Catal. Lett. 1991, 10, 181;
vi) Bellusi, G.; Conti, G.; Perathonar, S.; Trifiró, F. Proceedings, Symposium on Catalytic Selective Oxidation, Washington, D.C.; American Chemical Society: Washington, D.C., 1992; p 1242;
vii) Ind. Eng. Chem. Res. 1996, 35, 2137-2143 and
viii) Symposium on Heterogeneous Hydrocarbon Oxidation Presented before the Division of Petroleum Chemistry, Inc. 211th National Meeting, American Chemical Society New Orleans, La., Mar. 24-29, 1996.

Particularly suitable oxydehydrogenation catalysts are the multimetal oxide compositions or catalysts A of DE-A 1 97 53 817, and the multimetal oxide compositions or catalysts A specified as preferred are very particularly favorable. In other words, useful active compositions are in particular multimetal oxide compositions of the general formula I

M¹ₐMo_{1-b}M²_{b}Oₓ (I)

where
M¹=Co, Ni, Mg, Zn, Mn and/or Cu,
M²=W, V, Te, Nb, P, Cr, Fe, Sb, Ce, Sn and/or La,
a=from 0.5 to 1.5,
b=from 0 to 0.5 and
x=a number which is determined by the valency and frequency of the elements in I other than oxygen.

Further multimetal oxide compositions suitable as oxydehydrogenation catalysts are specified below:

Suitable Mo—V—Te/Sb—Nb—O multimetal oxide catalysts are disclosed in EP-A 0 318 295, EP-A 0 529 853, EP-A 0 603 838, EP-A 0 608 836, EP-A 0 608 838, EP-A 0 895 809, EP-A 0 962 253, EP-A 1 192 987, DE-A 198 35 247, DE-A 100 51 419 and DE-A 101 19 933.

Suitable Mo—V—Nb—O multimetal oxide catalysts are described, inter alia, in E. M. Thorsteinson, T. P. Wilson, F. G. Young, P. H. Kasei, Journal of Catalysis 52 (1978), pages 116-132, and in U.S. Pat. No. 4,250,346 and EP-A 0 294 845.

Suitable Ni—X—O multimetal oxide catalysts where X=Ti, Ta, Nb, Co, Hf, W, Y, Zn, Zr, Al, are described in WO 00/48971.

In principle, suitable active compositions can be prepared in a simple manner by obtaining from suitable sources of their components a very intimate, preferably finely divided dry mixture corresponding to the stoichiometry and calcining it at temperatures of from 450 to 1000° C. The calcination may be effected either under inert gas or under an oxidative atmosphere, for example air (mixture of inert gas and oxygen), and also under a reducing atmosphere (for example mixture of inert gas, oxygen and NH₃, CO and/or H₂). Useful sources for the components of the multimetal oxide active compositions include oxides and/or those compounds which can be converted to oxides by heating, at least in the presence of oxygen. In addition to the oxides, such useful starting compounds are in particular halides, nitrates, formates, oxalates, citrates, acetates, carbonates, amine complex salts, ammonium salts and/or hydroxides.

The multimetal oxide compositions may be used for the process according to the invention either in powder form or shaped to certain catalyst geometries, and this shaping may be effected before or after the final calcining. Suitable unsupported catalyst geometries are, for example, solid cylinders or hollow cylinders having an external diameter and a length of from 2 to 10 mm. In the case of the hollow cylinders, a wall thickness of from 1 to 3 mm is appropriate. The suitable hollow cylinder geometries are, for example, 7 mm×7 mm×4 mm or 5 mm×3 mm×2 mm or 5 mm×2 mm×2 mm (in each case length×external diameter×internal diameter). The unsupported catalyst can of course also have spherical geometry, in which case the sphere diameter may be from 2 to 10 mm.

The pulverulent active composition or its pulverulent precursor composition which is yet to be calcined may of course also be shaped by applying to preshaped inert catalyst supports. The layer thickness of the powder composition applied to the support bodies is appropriately selected within the range from 50 to 500 mm, preferably within the range from 150 to 250 mm. Useful support materials include customary porous or nonporous aluminum oxides, silicon dioxide, thorium dioxide, zirconium dioxide, silicon carbide or silicates such as magnesium silicate or aluminum silicate. The support bodies may have a regular or irregular shape, preference being given to regularly shaped support bodies having distinct surface roughness, for example spheres, hollow cylinders or saddles having dimensions in the range from 1 to 100 mm. It is suitable to use substantially nonporous, surface-rough, spherical supports of steatite whose diameter is from 1 to 8 mm, preferably from 4 to 5 mm.

The reaction temperature of the heterogeneously catalyzed oxydehydrogenation of propane is generally from 300 to 600° C., typically from 350 to 500° C. The pressure is from 0.2 to 15 bar, preferably from 1 to 10 bar, for example from 1 to 5 bar. Pressures above 1 bar, for example from 1.5 to 10 bar, have been found to be particularly advantageous. In general, the heterogeneously catalyzed oxydehydrogenation of propane is effected over a fixed catalyst bed. The latter is appropriately deposited in the tubes of a tube bundle reactor, as described, for example, in EP-A 700 893 and in EP-A 700 714 and the literature cited in these documents. The average residence time of the reaction gas mixture in the catalyst bed is normally from 0.5 to 20 sec. The propane to oxygen ratio in the starting reaction gas mixture to be used for the heterogeneously catalyzed propane oxydehydrogenation may, according to the invention, be from 0.5:1 to 40:1. It is advantageous when the molar ratio of propane to molecular oxygen in the starting gas mixture is ≦6:1, preferably ≦5:1. In general, the aforementioned ratio is ≧1:1, for example 2:1. The starting gas mixture may comprise further, substantially inert constituents such as H₂O, CO₂, CO, N₂, noble gases and/or propene. In addition, C₁, C₂ and C₄ hydrocarbons may also be comprised to a small extent.

When it leaves the dehydrogenation zone, the product gas stream b is generally under a pressure of from 0.2 to 15 bar, preferably from 1 to 10 bar, more preferably from 1 to 5 bar, and has a temperature in the range from 300 to 700° C.

In the propane dehydrogenation, a gas mixture is obtained which generally has the following composition: from 10 to 80% by volume of propane, from 5 to 50% by volume of propene, from 0 to 20% by volume of methane, ethane, ethene and C₄⁺ hydrocarbons, from 0 to 30% by volume of carbon oxides, from 0 to 70% by volume of steam, from 0 to 25% by volume of hydrogen, and from 0 to 50% by volume of inert gases.

In the preferred autothermal propane dehydrogenation, a gas mixture is obtained which generally has the following composition: from 10 to 80% by volume of propane, from 5 to 50% by volume of propene, from 0 to 20% by volume of methane, ethane, ethene and C₄⁺ hydrocarbons, from 0.1 to 30% by volume of carbon oxides, from 1 to 70% by volume of steam and from 0.1 to 25% by volume of hydrogen, and also from 0 to 30% by volume of inert gases.

In process part C), water is initially removed from the product gas stream b. The removal of water is carried out by condensation, by cooling and if appropriate compression of the product gas stream b, and may be carried out in one or more cooling and if appropriate compression stages. In general, the product gas stream b is cooled for this purpose to a temperature in the range from 20 to 80° C., preferably from 40 to 65° C. In addition, the product gas stream may be compressed, generally to a pressure in the range from 2 to 40 bar, preferably from 5 to 20 bar, more preferably from 10 to 20 bar.

In one embodiment of the process according to the invention, the product gas stream b is passed through a battery of heat exchangers and initially thus initially cooled to a temperature in the range from 50 to 200° C. and subsequently cooled further in a quenching tower with water to a temperature of from 40 to 80° C., for example 55° C. This condenses out the majority of the steam, but also some of the C₄⁺ hydrocarbons present in the product gas stream b, in particular the C₅⁺ hydrocarbons. Suitable heat exchangers are, for example, direct heat exchangers and countercurrent heat exchangers, such as gas-gas countercurrent heat exchangers, and air coolers.

A steam-depleted product gas stream c is obtained. This generally still comprises from 0 to 10% by volume of steam. For the virtually full removal of water from the product gas stream c when certain adsorbents are used in step D), a drying by means of molecular sieve, in particular 3A, 4A, 13X molecular sieve or preferably aluminum oxides, or membranes may be provided.

Before performing process step D), carbon dioxide can be removed from the gas stream c by gas scrubbing or by absorption on solid absorbents. The carbon dioxide gas scrubbing may precede a separate combustion stage in which carbon monoxide is oxidized selectively to carbon dioxide.

For CO₂ removal, the scrubbing liquid used is generally sodium hydroxide solution, potassium hydroxide solution or an alkanolamine solution; preference is given to using an activated N-methyldiethanolamine solution. In general, before the gas scrubbing is carried out, the product gas stream c is compressed by single-stage or multistage compression to a pressure in the range from 5 to 25 bar. A carbon dioxide-depleted stream c with a CO₂ content of generally <1000 ppm, preferably <100 ppm, more preferably <20 ppm can be obtained.

However, preference is given to removing CO₂ by sorption on suitable solid sorbents, for example 13X molecular sieve, calcium oxide, barium oxide, magnesium oxide or hydrotalcites.

In a process step D), the product gas stream c is contacted in an adsorption zone with a selective adsorbent which adsorbs propene selectively under the selected adsorption conditions to obtain a propene-laden adsorbent and a propene-depleted gas stream d2 comprising propane, methane, ethane, ethene, carbon monoxide, carbon dioxide and hydrogen. Propene may also be present in the gas stream d2.

In a desorption step E), a propene-comprising gas stream e1 is released from the adsorbent laden essentially with propene by pressure reduction and/or heating of the adsorbent. The pressure may be the total pressure and/or the partial pressure of propene specifically.

Suitable adsorbents are adsorbents comprising a porous metal-organic framework material (MOF). Further suitable adsorbents are molecular sieves, activated carbon, silica gel and xero- and aerogels, and also porous covalent organic framework materials (COF; A. P. Côté et al., Science 310 (2005), 1166 to 1170).

It has been found that especially porous metal-organic framework materials (MOF) bring about efficient separation of propene on the one hand, and propane and further gas constituents on the other hand.

The porous metal-organic framework materials comprise at least one at least bidentate organic compound bonded coordinatively to at least one metal ion. These metal-organic framework materials (MOF) are described, for example, in U.S. Pat. No. 5,648,508, EP-A-0 790 253, M. O-Keefe et al., J. Sol. State Chem., 152 (2000), page 3 to 20, H. Li et al., Nature 402 (1999), page 276, M. Eddaoudi et al., Topics in Catalysis 9, (1999), page 105 to 111, B. Chen et al., Science 291, (2001), page 1021 to 1023 and DE-A-101 11230, WO-A 2005/049 892 and A. C. Sudik et al., J Am. Chem. Soc. 127(2005), 7110 to 7118.

The metal-organic framework materials according to the present invention comprise pores, especially micro- and/or mesopores. Micropores are defined as those having a diameter of 2 nm or less and mesopores are defined by a diameter in the range from 2 to 50 nm, in each case corresponding to the definition as given by Pure Applied Chem. 45, page 71, in particular on page 79 (1976). The presence of micro- and/or mesopores can be tested with the aid of sorption measurements, these measurements determining the uptake capacity of the MOF for nitrogen at 77 Kelvin to DIN 66131 and/or DIN 66134.

The specific surface area, calculated by the Langmuir model to DIN 66135 (DIN 66131, 66134) for a framework material in powder form, is preferably more than 5 m²/g, more preferably more than 10 m²/g, more preferably more than 50 m²/g, even more preferably more than 500 m²/g, even more preferably more than 1000 m²/g and especially preferably more than 1500 m²/g.

MOF shaped bodies may have a lower active surface area; but preferably more than 10 m²/g, more preferably more than 50 m²/g, even more preferably more than 500 m²/g, in particular more than 1000 m²/g.

In the context of the present invention, the maximum of the pore diameter distribution should be at least 4 Å. This maximum is preferably between 4.3 and 20 Å. The range is more preferably between 5 and 13 Å.

The metal component in the framework material according to the present invention is preferably selected from groups Ia, IIa, IIIa, IVa to VIIIa and Ib to VIb. Preference is further given to groups IIa, IIIb, IIIa to VIa of the periodic table of the elements, and to the lanthanides, V, Mn, Fe, Ni, Co. Particular preference is given to Mg, Ca, Sr, Ba, Sc, Y, Ti, Zr, Hf, V, Nb, Ta, Cr, Mo, W, Mn, Re, Fe, Ro, Os, Co, Rh, Ir, Ni, Pd, Pt, Cu, Ag, Au, Zn, Cd, Hg, Al, Ga, In, Tl, Si, Ge, Sn, Pb, As, Sb and Bi. Also more preferred are Mg, Al, In, Cu, Zn, Fe, Ni, Co, Mn, Zr, Ti, Sc, Y, La, Ce. More preferred are Mg, Al, In, Cu, Zn, Fe, Zr, Y. In the case of copper, preference is given to MOF types which have no free Cu coordination sites.

In relation to the ions of these elements, particular mention should be made of Mg²⁺, Ca²⁺, Sr²⁺, Ba²⁺, Sc³⁺, Y³⁺, Ti⁴⁺, Zr⁴⁺, Hf⁴⁺, V⁴⁺, V³⁺, Nb³⁺, Ta³⁺, Cr³⁺, Mo³⁺, W³⁺, Mn³⁺, Mn²⁺, Re³⁺, Re²⁺, Fe³⁺, Fe²⁺, Ru³⁺, Os³⁺, Os²⁺, Co³⁺, Co²⁺, Rh²⁺, Rh⁺, Ir²⁺, Ir⁺, Ni²⁺, Ni⁺, Pd²⁺, Pd⁺, Pt²⁺, Pt⁺, Cu²⁺, Cu⁺, Ag⁺, Au⁺, Zn²⁺, Cd²⁺, Hg²⁺, Al³⁺, Ga³⁺, In³⁺, Ti³⁺, Si⁴⁺, Si²⁺, Ge⁴⁺, Ge²⁺, Sn⁴⁺, Sn²⁺, Pb⁴⁺, Pb²⁺, Pb²⁺, As⁵⁺, As³⁺, As⁺, Sb⁵⁺, Sb³⁺, Sb⁺, Bi⁵⁺, Bi³⁺, and Bi⁺.

The expression “at least bidentate organic compound” refers to an organic compound which comprises at least one functional group which is capable of forming at least two, preferably two, coordinate bonds to a given metal ion, and/or in each case one coordinate bond to two or more, preferably two, metal atoms.

Functional groups through which the coordinate bonds mentioned can be formed are in particular, for example, the following functional groups: —CO₂H, —CS₂H, —NO₂, —B(OH)₂, —SO₃H, —Si(OH)₃, —Ge(OH)₃, —Sn(OH)₃, —Si(SH)₄, —Ge(SH)₄, —Sn(SH)₃, —PO₃H, —AsO₃H, —AsO₄H, —P(SH)₃, —As(SH)₃, —CH(RSH)₂, —C(RSH)₃, —CH(RNH₂)₂, —C(RNH₂)₃, —CH(ROH)₂, —C(ROH)₃, —CH(RCN)₂, —C(RCN)₃, where R is, for example and with preference, an alkylene group having 1, 2, 3, 4 or 5 carbon atoms, for example a methylene, ethylene, n-propylene, i-propylene, n-butylene, i-butylene, tert-butylene or n-pentylene group, or an aryl group comprising 1 or 2 aromatic rings, for example 2 C₆ rings, which may, if appropriate, be condensed and each independently suitably be substituted by at least one substituent in each case, and/or which may each independently comprise at least one heteroatom, for example N, O and/or S. According to likewise preferred embodiments, functional groups to be mentioned are those in which the abovementioned R radical is not present. In this regard, mention should be made, inter alia, of —CH(SH)₂, —C(SH)₃, —CH(NH₂)₂, —C(NH₂)₃, —CH(OH)₂, —C(OH)₃, CH(CN)₂ or —C(CN)₃.

The at least two functional groups may in principle be bonded to any suitable organic compound, provided that it is ensured that the organic compound having these functional groups is capable of forming the coordinate bond and of producing the framework material.

The organic compounds which comprise the at least two functional groups preferably derive from a saturated or unsaturated aliphatic compound or an aromatic compound or a both aliphatic and aromatic compound.

The aliphatic compound or the aliphatic moiety of the both aliphatic and aromatic compound may be linear and/or branched and/or cyclic, and a plurality of cycles per compound is also possible. More preferably, the aliphatic compound or the aliphatic moiety of the both aliphatic and aromatic compound comprises from 1 to 15, more preferably from 1 to 14, more preferably from 1 to 13, more preferably from 1 to 12, more preferably from 1 to 11 and especially preferably from 1 to 10 carbon atoms, for example 1, 2, 3, 4, 5, 6, 7, 8, 9 or 10 carbon atoms. Especially preferred here are, inter alia, methane, adamantane, acetylene, ethylene or butadiene.

The aromatic compound or the aromatic moiety of the both aromatic and aliphatic compound may have one or even more rings, for example two, three, four or five rings, in which case the rings may be present separately from one another and/or at least two rings may be present in fused form. The aromatic compound or the aromatic moiety of the both aliphatic and aromatic compound more preferably has one, two or three rings, particular preference being given to one or two rings. Independently of one another, every ring of the compound mentioned may also comprise at least one heteroatom, for example N, O, S, B, P, Si, Al, preferably N, O and/or S. The aromatic compound or the aromatic moiety of the both aromatic and aliphatic compound more preferably comprises one or two C₆ rings, in which the two are present separately from one another or in fused form. Aromatic compounds of which particular mention should be made are benzene, naphthalene and/or biphenyl and/or bipyridyl and/or pyridyl.

For example, mention should be made, inter alia, of trans-muconic acid or fumaric acid or phenylenebisacrylic acid.

The at least bidentate organic compound preferably derives from a di-, tri- or tetracarboxylic acid or its sulfur analogs. Sulfur analogs are the functional groups —C(═O)SH and its tautomer, and C(═S)SH, which may be used instead of one or more carboxylic acid groups.

In the context of the present invention, the term “derive” means that the at least bidentate organic compound may be present in the framework material in partly deprotonated or fully deprotonated form. In addition, the at least bidentate organic compound may comprise further substituents, for example —OH, —NH₂, —OCH₃, —CH₃, —NH(CH₃), —N(CH₃)₂, —CN and halides.

For example, mention should be made in the context of the present invention of dicarboxylic acids such as

oxalic acid, succinic acid, tartaric acid, 1,4-butanedicarboxylic acid, 4-oxopyran-2,6-dicarboxylic acid, 1,6-hexanedicarboxylic acid, decanedicarboxylic acid, 1,8-heptadecanedicarboxylic acid, 1,9-heptadecanedicarboxylic acid, heptadecanedicarboxylic acid, acetylenedicarboxylic acid, 1,2-benzenedicarboxylic acid, 2,3-pyridinedicarboxylic acid, pyridine-2,3-dicarboxylic acid, 1,3-butadiene-1,4-dicarboxylic acid, 1,4-benzenedicarboxylic acid, p-benzenedicarboxylic acid, imidazole-2,4-dicarboxylic acid, 2-methylquinoline-3,4-dicarboxylic acid, quinoline-2,4-dicarboxylic acid, quinoxaline-2,3-dicarboxylic acid, 6-chloroquinoxaline-2,3-dicarboxylic acid, 4,4′-diaminophenylmethane-3,3′-dicarboxylic acid, quinoline-3,4-dicarboxylic acid, 7-chloro-4-hydroxyquinoline-2,8-dicarboxylic acid, diimidedicarboxylic acid, pyridine-2,6-dicarboxylic acid, 2-methylimidazole-4,5-dicarboxylic acid, thiophene-3,4-dicarboxylic acid, 2-isopropylimidazole-4,5-dicarboxylic acid, tetrahydropyran-4,4′-dicarboxylic acid, perylene-3,9-dicarboxylic acid, perylenedicarboxylic acid, Pluriol E 200-dicarboxylic acid, 3,6-dioxaoctanedicarboxylic acid, 3,5-cyclohexadiene-1,2-dicarboxylic acid, octadicarboxylic acid, pentane-3,3-dicarboxylic acid, 4,4′-diamino- 1,1′-biphenyl-3,3′-dicarboxylic acid, 4,4′-diaminobiphenyl-3,3′-dicarboxylic acid, benzidine-3,3′-dicarboxylic acid, 1,4-bis(phenylamino)benzene-2,5-dicarboxylic acid, 1,1′-dinaphthyldicarboxylic acid, 7-chloro-8-methylquinoline-2,3-dicarboxylic acid, 1-anilinoanthraquinone-2,4′-dicarboxylic acid, polytetrahydrofuran-250-dicarboxylic acid, 1,4-bis(carboxymethyl)piperazine-2,3-dicarboxylic acid, 7-chloroquinoline-3,8-dicarboxylic acid, 1-(4-carboxy)phenyl-3-(4-chloro)phenylpyrazoline-4,5-dicarboxylic acid, 1,4,5,6,7,7-hexachloro-5-norbornene-2,3-dicarboxylic acid, phenylindanedicarboxylic acid, 1,3-dibenzyl-2-oxoimidazolidine-4,5-dicarboxylic acid, 1,4-cyclohexanedicarboxylic acid, naphthalene-1,8-dicarboxylic acid, 2-benzoylbenzene-1,3-dicarboxylic acid, 1,3-dibenzyl-2-oxoimidazolidine4,5-cis-dicarboxylic acid, 2,2′-biquinoline-4,4′-dicarboxylic acid, pyridine-3,4-dicarboxylic acid, 3,6,9-trioxaundecanedicarboxylic acid, O-hydroxybenzophenonedicarboxylic acid, Pluriol E 300-dicarboxylic acid, Pluriol E 400-dicarboxylic acid, Pluriol E 600-dicarboxylic acid, pyrazole-3,4-dicarboxylic acid, 2,3-pyrazinedicarboxylic acid, 5,6-dimethyl-2,3-pyrazinedicarboxylic acid, (bis(4-aminophenyl) ether)diimidedicarboxylic acid, 4,4′-diaminodiphenylmethanediimidedicarboxylic acid, (bis(4-aminophenyl) sulfone)diimidedicarboxylic acid, 2,6-naphthalenedicarboxylic acid, 1,3-adamantanedicarboxylic acid, 1,8-naphthalenedicarboxylic acid, 2,3-naphthalenedicarboxylic acid, 8-methoxy-2,3-naphthalenedicarboxylic acid, 8-nitro-2,3-naphthalenecarboxylic acid, 8-sulfo-2,3-naphthalenedicarboxylic acid, anthracene-2,3-dicarboxylic acid, 2′,3′-diphenyl-p-terphenyl-4,4″-dicarboxylic acid, (diphenyl ether)-4,4′-dicarboxylic acid, imidazole-4,5-dicarboxylic acid, 4(1H)-oxothiochromene-2,8-dicarboxylic acid, 5-tert-butyl-1,3-benzenedicarboxylic acid, 7,8-quinolinedicarboxylic acid, 4,5-imidazoledicarboxylic acid, 4-cyclohexene-1,2-dicarboxylic acid, hexatriacontanedicarboxylic acid, tetradecanedicarboxylic acid, 1,7-heptadicarboxylic acid, 5-hydroxy-1,3-benzenedicarboxylic acid, pyrazine-2,3-dicarboxylic acid, furan-2,5-dicarboxylic acid, 1-nonene-6,9-dicarboxylic acid, eicosenedicarboxylic acid, 4,4′-dihydroxydiphenylmethane-3,3′-dicarboxylic acid, 1-amino-4-methyl-9,10-dioxo-9,10-dihydroanthracene-2,3-dicarboxylic acid, 2,5-pyridinedicarboxylic acid, cyclohexene-2,3-dicarboxylic acid, 2,9-dichlorofluorubin-4,11-dicarboxylic acid, 7-chloro-3-methylquinoline-6,8-dicarboxylic acid, 2,4-dichlorobenzophenone-2′,5′-dicarboxylic acid, 1,3-benzenedicarboxylic acid, 2,6-pyridinedicarboxylic acid, 1-methylpyrrole-3,4-dicarboxylic acid, 1-benzyl-1H-pyrrole-3,4-dicarboxylic acid, anthraquinone-1,5-dicarboxylic acid, 3,5-pyrazoledicarboxylic acid, 2-nitrobenzene- 1,4-dicarboxylic acid, heptane-1,7-dicarboxylic acid, cyclobutane-1,1-dicarboxylic acid, 1,14-tetradecanedicarboxylic acid, 5,6-dehydronorbornane-2,3-dicarboxylic acid or 5-ethyl-2,3-pyridinedicarboxylic acid, tricarboxylic acids such as
2-hydroxy-1,2,3-propanetricarboxylic acid, 7-chloro-2,3,8-quinolinetricarboxylic acid, 1,2,4-benzenetricarboxylic acid, 1,2,4-butanetricarboxylic acid, 2-phosphono-1,2,4-butanetricarboxylic acid, 1,3,5-benzenetricarboxylic acid, 1-hydroxy-1,2,3-propanetricarboxylic acid, 4,5-dihydro-4,5-dioxo-1H-pyrrolo[2,3-F]quinoline-2,7,9-tricarboxylic acid, 5-acetyl-3-amino-6-methylbenzene-1,2,4-tricarboxylic acid, 3-amino-5-benzoyl-6-methylbenzene-1,2,4-tricarboxylic acid, 1,2,3-propanetricarboxylic acid or aurintricarboxylic acid, or tetracarboxylic acids such as
1,1-dioxidoperylo[1,12-BCD]thiophene-3,4,9,10-tetracarboxylic acid, perylenetetra-carboxylic acids such as perylene-3,4,9,10-tetracarboxylic acid or (perylene 1,12-sulfone)-3,4,9,10-tetracarboxylic acid, butanetetracarboxylic acids such as 1,2,3,4-butanetetracarboxylic acid or meso-1,2,3,4-butanetetracarboxylic acid, decane-2,4,6,8-tetracarboxylic acid, 1,4,7,10,13,16-hexaoxacyclooctadecane-2,3,11,12-tetracarboxylic acid, 1,2,4,5-benzenetetracarboxylic acid, 1,2,11,12-dodecanetetracarboxylic acid, 1,2,5,6-hexanetetracarboxylic acid, 1,2,7,8-octanetetracarboxylic acid, 1,4,5,8-naphthalenetetracarboxylic acid, 1,2,9,10-decanetetracarboxylic acid, benzophenonetetracarboxylic acid, 3,3′,4,4′-benzophenonetetracarboxylic acid, tetrahydrofurantetracarboxylic acid or cyclopentanetetracarboxylic acids such as cyclopentane-1,2,3,4-tetracarboxylic acid.

Very particular preference is given to using optionally at least monosubstituted, mono-, di-, tri- or tetracyclic, aromatic di-, tri- or tetracarboxylic acids where each of the rings may comprise at least one heteroatom and where two or more rings may comprise identical or different heteroatoms. For example, preference is given to monocyclic dicarboxylic acids, monocyclic tricarboxylic acids, monocyclic tetracarboxylic acids, bicyclic dicarboxylic acids, bicyclic tricarboxylic acids, bicyclic tetracarboxylic acids, tricyclic dicarboxylic acids, tricyclic tricarboxylic acids, tricyclic tetracarboxylic acids, tetracyclic dicarboxylic acids, tetracyclic tricarboxylic acids and/or tetracyclic tetracarboxylic acids. Suitable heteroatoms are, for example, N, O, S, B, P, Si; preferred heteroatoms here are N, S and/or O. Suitable substituents which may be mentioned in this respect include —OH, a nitro group, an amino group or an alkyl or alkoxy group.

The at least bidentate organic compounds used are especially preferably acetylenedicarboxylic acid (ADC), benzenedicarboxylic acids, naphthalenedicarboxylic acids, biphenyldicarboxylic acids such as 4,4′-biphenyldicarboxylic acid (BPDC), bipyridinedicarboxylic acids such as 2,2′-bipyridinedicarboxylic acids such as 2,2′-bipyridine-5,5′-dicarboxylic acid, benzenetricarboxylic acids such as 1,2,3-benzenetricarboxylic acid or 1,3,5-benzenetricarboxylic acid (BTC), adamantanetetracarboxylic acid (ATC), adamantanedibenzoate (ADB), benzenetribenzoate (BTB), methanetetrabenzoate (MTB), adamantanetetrabenzoate or dihydroxyterephthalic acids such as 2,5-dihydroxyterephthalic acid (DHBDC).

Very particular preference is given to using, inter alia, isophthalic acid, terephthalic acid, 2,5-dihydroxyterephthalic acid, 1,2,3-benzenetricarboxylic acid, 1,3,5-benzenetricarboxylic acid, 2,2′-bipyridine-5,5′-dicarboxylic acid, aminoterephthalic acid or diaminoterephthalic acid.

In addition to these at least bidentate organic compounds, the MOF may also comprise one or more monodentate ligands.

Suitable solvents for preparing the MOF include ethanol, dimethylformamide, toluene, methanol, chlorobenzene, diethylformamide, dimethyl sulfoxide, water, hydrogen peroxide, methylamine, sodium hydroxide solution, N-methylpyrrolidone ether, acetonitrile, benzyl chloride, triethylamine, ethylene glycol and mixtures thereof. Further metal ions, at least bidentate organic compounds and solvents for the preparation of MOF are described, inter alia, in U.S. Pat. No. 5,648,508 or DE-A 101 11 230.

The pore size of the MOF can be controlled by selection of the suitable ligand and/or of the at least bidentate organic compound. It is generally the case that the larger the organic compound, the larger the pore size. The pore size is preferably from 0.2 nm to 30 nm; the pore size is more preferably in the range from 0.3 nm to 3 nm based on the crystalline material.

However, larger pores also occur in an MOF shaped body and their size distribution may vary. However, preferably more than 50% of the total pore volume, in particular more than 75%, is formed by pores having a diameter of up to 1000 nm. However, a majority of the pore volume is formed by pores from two diameter ranges. It is therefore further preferred when more than 25% of the total pore volume, in particular more than 50% of the total pore volume, is formed by pores in a diameter range of from 100 nm to 800 nm and when more than 15% of the total pore volume, in particular more than 25% of the total pore volume, is formed by pores which are within a diameter range of up to 10 nm. The pore distribution can be determined by means of mercury porosimetry.

Examples of very particularly suitable MOFs are Cu-BTC (BTC=1,3,5-benzenetricarboxylic acid), Al-terephthalic acid, Cu-terephthalic acid-TEDA, Zn-terephthalic acid (MOF-5), Zn-terephthalic acid-TEDA, MOF-74, Zn-naphthalene-DC (IRMOF-8), Al-aminoterephthalic acid.

The metal-organic framework materials are generally used in the form of shaped bodies, for example as random packings of spheres, rings, extrudates or tablets, or as structured internals such as structured packings, honeycombs and monoliths.

The production of shaped bodies is described, for example, in WO-A 03/102 000. Preference is given to the use of random packings which are in very tightly packed form. The shaped bodies therefore have, at their narrowest point, a diameter of preferably not more than 3 mm, more preferably not more than 2 mm, most preferably not more than 1.5 mm. Very particular preference is given to shaped bodies in tablet form. An alternative is incorporation in the form of a monolithic structure, since the large channels can likewise be flushed readily here, while the material in the walls is likewise in very tightly packed form.

Suitable molecular sieves are described, for example, in C. A. Grande, A. E. Rodrigues, Ind. Eng. Chem. Res.; Propane-Propylene Separation by Pressure Swing Adsorption Using Zeolite 4A, 2005, 44, 8815-8829. A preferred molecular sieve is a 4A molecular sieve. In general, the 4A molecular sieve is laden at temperatures of at least 70° C., preferably at least 90° C. and in particular at least 100° C. In this case, propene with a purity of >90% or even >99% can be achieved.

Further suitable molecular sieves are described in:
C. A. Grande, S. Cavenati, F. Da Silva, A. E. Rodrigues, Ind. Eng. Chem. Res.; Carbon Molecular Sieves for Hydrocarbon Separations by Adsorption, 2005, 44, 7218-7227;
F. A. Da Silva, A. E. Rodrigues, AIChE Journal; Propane-Propylene Separation by Using 13X Zeolite, 2001, 47, 341-357;
F. A. Da Silva, Rodrigues, Ind. Eng. Chem. Res.; Vacuum Swing Adsorption for Propane-Propylene Separation with Zeolite 4A, 2001, 40, 5758-5774;
I. Giannakopoulus, V. Nikolakis, Ind. Eng. Chem. Res.; “Separation of Propylene/Propane Mixtures Using Faujasite-Type Zeolite Membranes” 2005, 44, 226-230;
J. Padin, S. Rege, R. Yang, L. Cheng, Chem. Eng. Science; “Molecular sieve sorbents for kinetic separation of propane/propylene” 2000, 55, 4525.

Particularly preferred molecular sieves are 4A, 5A, 13X.

Molecular sieves are generally used in the form of shaped bodies. Suitable shaped bodies are random packings of, for example, spheres, rings, extrudates and tablets, and also structured internals composed of structured packings, honeycombs and monoliths.

In the adsorption stage D), full removal of the propene from the remaining gas constituents is not required, since the gas stream d2 is recycled back into the propane dehydrogenation. The aim is maximum loading of the adsorbent with pure propene. Since the adsorption coefficient of propene on the adsorbent is higher than that of the other gas constituents, other gas constituents are gradually displaced from the adsorption sites, so that propene is finally adsorbed selectively.

For the performance of the adsorption stage D) and of the desorption stage E), a series of different possible embodiments are available to the person skilled in the art. What is common to all is that at least two, preferably three, more preferably at least four adsorbers are operated in parallel, of which at least two, but preferably all, work in a phase offset with respect to the other adsorber in each case. Possible variants are a) a pressure-swing adsorption (PSA), b) a vacuum pressure-swing adsorption (VPSA), c) a temperature-swing adsorption (TSA) or a combination of different processes. These processes are known in principle to the person skilled in the art and can be looked up in textbooks, for example W. Kast, “Adsorption aus der Gasphase—Ingenieurwissenschaftliche Grundlagen und technische Verfahren”, VCH Weinheim, 1988, D. M. Ruthven, S. Farooq, K. S. Knaebel, “Pressure Swing Adsorption”, Wiley-VCH, New York-Chichester-Weinheim-Brisbane-Singapore-Toronto, 1994 or D. Bathen, M. Breitbach, “Adsorptiontechnik”, Springer Verlag Berlin-Heidelberg, 2001, D. Basmadjian, “The Little Adsorption Book”, CRC Press Boca Raton, 1996 or publications, for example A. Mersmann, B. Fill, R. Hartmann, S. Maurer, Chem. Eng. Technol. 23/11 (2000) 937. The bed of an adsorber need not necessarily comprise only a single adsorbent, but may consist of a plurality of layers of different materials. This can be utilized, for example, in order to sharpen the breakthrough front of the adsorbed species during the adsorption phase.

For example, a pressure-swing adsorption for the propane/propene separation can be configured as follows: four reactors work in parallel in the following offset phases: in phase 1, an adsorber is brought to the working pressure (pₘₐₓᵢₘᵤₘ) by supplying gas from a second adsorber in adsorption mode or offgas from a second adsorber which is decompressed simultaneously, and fresh gas. In phase 2, the adsorbent is laden fully with propene by further feeding, preferably until the entire the entire adsorption front has been broken through and no further propene is adsorbed. In this case, before the propene front breaks through, a second reactor is preferably connected upstream in adsorption mode. In phase 3, the adsorber is flushed with pure propene in order to displace unadsorbed residual propane present in the adsorber. The flushing can be effected in co- or countercurrent, preference being given to cocurrent. The flushing can be effected at adsorption pressure. To save pure propene, however, preference is given to an earlier lowering of the adsorber pressure; particular preference is given to a similar partial propene pressure in the adsorption phase (phase 2) and flush phase (phase 3). The gas mixture released in the course of this pressure lowering can be fed to another adsorber during phase 1 for pressure buildup. In phase 4, the laden and flushed adsorber is decompressed to obtain the pure propylene stream. The product is preferably removed in countercurrent.

In addition, a reduced pressure can be applied in phase 4. This embodiment is an example of a VPSA process.

To compensate for the temperature effects owing to the heat of adsorption/cold of desorption, the supply or removal of heat may be advantageous. The heat input can be effected in various ways: conductively via internal heat exchangers, convectively via external heat exchangers or by means of radiation, for example by means of incident microwaves or radio waves. It is likewise possible to utilize heat input over and above the compensation of the cold of desorption to additionally facilitate the desorption of the propene during phase 4. Such a process constitutes a combination of a pressure-swing adsorption and of a temperature-swing adsorption.

The product of value can also be desorbed by displacement with an auxiliary component, for example N₂, CO₂ or steam. This utilizes the fact that the auxiliary component lowers the partial pressure of the propylene in the gas phase, while the absolute pressure can remain constant. In addition, a more strongly adsorbing auxiliary component, for example steam or CO₂, can also lead to a displacement of the product of value from the surface of the adsorbent. In the latter case, however, the auxiliary component must, in a further step, be removed again from the surface of the adsorbent, for example by raising the temperature. In this case, for example, temperature levels can also be set which, in the presence of propylene, lead to undesired side reactions, for example polymerization. Since the auxiliary can get into the desorbed product of value in such a method, a removal step, for example by condensation, adsorption, separation via a membrane, distillation or by selective scrubbing, may follow.

The phases need not necessarily last for the same time, so that a smaller or larger number of adsorbers may also be used for synchronization.

If the desorbed propene does not have the desired purity, a further purification, preferably by adsorption, may follow, in which case a different adsorbent may also be used here.

The adsorption is generally carried out at a temperature in the range from −50 to 250° C., preferably from 10 to 100° C. and more preferably from 10 to 50° C. The adsorption in the case of use of molecular sieves is preferably carried out within the range from 100 to 150° C.; in the case of use of MOFs, at from −50 to 100° C.

The adsorption is effected at a pressure in the range from 1 to 40 bar, preferably from 1.5 to 20 bar, more preferably from 2 to 15 bar and in particular from 2.5 to 10 bar.

The desorption phase itself can be effected by lowering the (partial) pressure or by heat input or by a combination of the two measures.

The adsorption/desorption can be configured as a fixed bed process, fluidized bed process or moving bed process. Examples of suitable apparatus are fixed bed reactors, rotary adsorbers or blind filters. A comprehensive description of possible apparatus can be found in: Werner Kast, “Adsorption aus der Gasphase”, VCH (Weinheim); H. Brauer, “Die Adsorptionstechnik, Ein Gebiet mit Zukunft”, Chem.-Ing. Tech 57 (1985) 8, 650-653; Dieter Bathen, Marc Breitbach “Adsorptionstechnik”, VDI-Buch, 2001.

To desorb the gases adsorbed in the adsorbent, it is heated and/or decompressed to a lower pressure.

The propene-comprising gas stream el released by desorption comprises generally, based on the hydrocarbon content, at least 90% by volume of propene, preferably at least 95% by volume of propene, more preferably at least 99.5% by volume of propene. In addition, it may comprise from 0 to 5% by volume of propane and also small amounts of CO, CO₂, ethane, ethene and methane, but generally not more than 1% by volume, preferably not more than 0.5% by volume. When a displacement desorption is performed, the stream e1 may additionally comprise the flushing gas, for example CO₂.

Depending on the adsorbent used, for example for Cu-containing metal-organic framework materials such as Cu-BTC, a selective hydrogenation can be performed to remove acetylenes and allenes, which in some cases are adsorbed better to the adsorbent than propene, before performing the adsorption stage D). The acetylene content in the stream c should generally be <1′%, preferably <500 ppm, more preferably <100 ppm, in particular <10 ppm. The selective hydrogenation may be required if significant amounts of acetylenes and allenes (methylacetylene and propadiene) are formed in the propane dehydrogenation. The selective hydrogenation may be carried out with externally supplied hydrogen or hydrogen present in the product gas stream of the dehydrogenation.

In one embodiment of the process according to the invention, the propane-comprising gas stream d2 is recycled at least partly directly into the dehydrogenation zone, and a substream (purge gas stream) is generally removed from the gas stream d2 to discharge inert gases, hydrogen and carbon oxides. The purge gas stream can be incinerated. However, it is also possible to recycle one substream of the gas stream d2 directly into the dehydrogenation zone, and to remove propane from a further substream by absorption and desorption and to recycle it into the dehydrogenation zone.

In a further preferred embodiment of the process according to the invention, at least a portion of the propane-comprising gas stream d2 obtained in step D) is contacted in a further step F) with a high-boiling absorbent, and the gases dissolved in the absorbent are subsequently desorbed to obtain a recycle stream f1 consisting essentially of propane and an offgas stream f2 comprising methane, ethane, ethene and hydrogen, if appropriate carbon monoxide and carbon dioxide. The recycle stream consisting essentially of propane is recycled into the first dehydrogenation zone.

To this end, in an absorption stage, the gas stream d2 is contacted with an inert absorbent, and propane and also small amounts of the C₂ hydrocarbons are absorbed in the inert absorbent to obtain an absorbent laden with propane and an offgas comprising the remaining gas constituents. These are essentially carbon oxides, hydrogen, inert gases, and also C₂ hydrocarbons and methane. In a desorption stage, propane is released again from the absorbent.

Inert absorbents used in the absorption stage are generally high-boiling nonpolar solvents in which the propane to be removed has a distinctly higher solubility than the remaining gas substituents. The absorption can be effected by simply passing stream d2 through the absorbent. However, it can also be effected in columns or in rotary absorbers. It is possible to work in cocurrent, countercurrent or crosscurrent. Suitable absorption columns are, for example, tray columns having bubble-cap, centrifugal and/or sieve trays, columns having structured packings, for example fabric packings or sheet metal packings having a specific surface area of from 100 to 1000 m²/m³, such as Mellapak® 250 Y, and columns having random packing. However, useful absorption apparatus also includes trickle and spray towers, graphite block absorbers, surface absorbers such as thick-film and thin-film absorbers, and rotary columns, pan scrubbers, cross-spray scrubbers, rotary scrubbers and bubble columns with and without internals.

Suitable absorbents are comparatively nonpolar organic solvents, for example aliphatic C₄-C₁₈-alkenes, naphtha or aromatic hydrocarbons such as the middle oil fractions from paraffin distillation, or ethers having bulky groups, or mixtures of these solvents, to each of which a polar solvent such as dimethyl 1,2-phthalate may be added. Further suitable absorbents are esters of benzoic acid and phthalic acid with straight-chain C₁-C₈-alkanols, such as n-butyl benzoate, methyl benzoate, ethyl benzoate, dimethyl phthalate, diethyl phthalate, and also heat carrier oils such as biphenyl and diphenyl ether, their chlorine derivatives, and also triarylalkenes. A suitable absorbent is a mixture of biphenyl and diphenyl ether, preferably in the azeotropic composition, for example the commercially available Diphyl®. This solvent mixture frequently comprises dimethyl phthalate in an amount of from 0.1 to 25% by weight. Suitable absorbents are also butanes, pentanes, hexanes, heptanes, octanes, nonanes, decanes, undecanes, dodecanes, tridecanes, tetradecanes, pentadecanes, hexadecanes, heptadecanes and octadecanes, or fractions which are obtained from refinery streams and comprise the linear alkanes mentioned as main components.

To desorb the propane, the laden absorbent is heated and/or decompressed to a lower pressure. Alternatively, the desorption can also be effected by stripping, typically with steam or an oxygenous gas, or in a combination of decompression, heating and stripping, in one or more process steps. For example, the desorption may be carried out in two stages, in which case the second desorption stage is carried out at a lower pressure than the first desorption stage and the desorption gas of the first stage is recycled into the absorption stage. The absorbent regenerated in the desorption stage is recycled into the absorption stage.

In one process variant, the desorption step is carried out by decompressing and/or heating the laden absorbent. In a further process variant, stripping is effected additionally with steam. In a further process variant, stripping is effected additionally with an oxygenous gas. The amount of the stripping gas used may correspond to the oxygen demand of the autothermal dehydrogenation.

Alternatively, an adsorption/desorption with a fixed bed adsorbent may also be carried out to remove propane from the remaining gas constituents to obtain a recycle stream f1 consisting essentially of propane.

Alternatively, in process step F), carbon dioxide can be removed by gas scrubbing from the gas stream d2 or a substream thereof to obtain a carbon dioxide-depleted recycle stream f1. The carbon dioxide gas scrubbing may precede a separate combustion stage in which carbon monoxide is oxidized selectively to carbon dioxide.

For CO₂ removal, the scrubbing liquid used is generally sodium hydroxide solution, potassium hydroxide solution or an alkanolamine solution; preference is given to using an activated N-methyldiethanolamine solution. In general, before the gas scrubbing is carried out, the product gas stream c is compressed by single-stage or multistage compression to a pressure in the range from 5 to 25 bar. It may obtained a carbon dioxide-depleted recycle stream f1 with a CO₂ content of generally <100 ppm, preferably <10 ppm.

Hydrogen may, if appropriate, be removed from the gas stream d2 by membrane separation or pressure-swing absorption.

To remove the hydrogen present in the offgas stream, it may, if appropriate after it has been cooled, for example, be passed in an indirect heat exchanger through a membrane, generally configured as a tube, which is permeable only to molecular hydrogen. The thus removed molecular hydrogen may, if required, be used at least partly in the dehydrogenation or else sent to another utilization, for example to the generation of electrical energy in fuel cells. Alternatively, the offgas stream d2 may be incinerated.

An adsorptive workup can be effected alternatively or additionally to the absorption in step F).

The invention is illustrated in detail by the example which follows.

### EXAMPLE

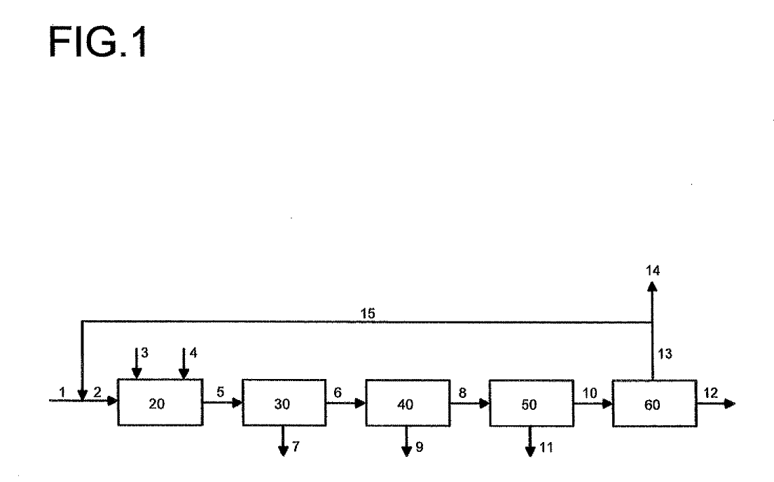

The variant, shown in FIG. 1, of the process according to the invention was simulated by calculation. In this simulation, a propane conversion in the dehydrogenation stage of 35%, a selectivity for propene of 95.4% and additionally the formation of 2.3% crack products and 2.3% combustion products were assumed. A capacity of the plant of 350 kt/a of propene at a running time of 8000 h/a was assumed.

A propane-containing feed stream (1) which had been freed beforehand of low boilers (C₄⁺ hydrocarbons) in a depropanizer (in the example, the feed stream (1) still comprises 0.01% by weight of residual C₄ hydrocarbon), is combined with the recycle streams (15), preheated to 450° C. in the heater and fed at approx. 8 bar as stream (2) to the autothermal PDH (20). To ensure autothermicity, steam (4) and pure oxygen (3) are also added thereto. The product gas (5) is cooled and fed to a multistage compression with intermediate coolings (30). This is effected starting from a pressure of 2.5 bar over 2 stages with turbocompressors to 10 bar. In the intermediate coolings at 55° C. with air condensers and heat exchangers, condensate is obtained which consists essentially of water (7) and is discharged from the process. Depending on the acetylene content of the PDH product gas (5), it is fed before the compression (30) first to a selective hydrogenation in which the acetylenes are hydrogenated to olefins with the dehydrogenation hydrogen present in the gas and, if appropriate, external hydrogen. The compressed gas (6) is fed first to a CO₂ scrubbing (40) before the adsorptive removal of the propene. For example by means of an activated MDEA scrubbing, the depletion of the CO₂ in stream 8 is effected here down to 30 ppm by weight. The CO₂ (9) released in the desorption is discharged from the process.

The stream (8) freed of CO₂ is, after further cooling and removal of the condensate, dried virtually fully by adsorption by means of a 4A molecular sieve in stage (50) (the stream 10 still comprises 10 ppm by weight of water). The stream 10 freed largely of CO₂ and water is then fed to the adsorption stage (60) in which the propene is removed as polymer-grade propene (12). The propene-depleted gas stream (13) (yield of the adsorption stage 90%) is divided. The predominant portion (15) is recycled directly to the PDH (20); a small purge stream (14) is removed from the process in order to discharge secondary components and hydrogen. The stream (14) can either be incinerated or a recovery of the propane by means of absorption or adsorption can be carried out.

The composition of the streams in parts by mass is reproduced by the table which follows.

TABLE

| Stream No. | 1 | 2 | 3 | 4 | 5 | 6 | 7 | 8 |
|---|---|---|---|---|---|---|---|---|
| Amount [kg/h] | 57575.1553 | 164871.992 | 16211.9046 | 13852.1998 | 194935.191 | 167077.774 | 27857.4151 | 164866.241 |
| BUTANE | 0.0001 | 0.00015 | 0 | 0 | 0.000127 | 0.000126 | 0.00013068 | 0.000128 |
| PROPANE | 0.9999 | 0.840179 | 0 | 0 | 0.461893 | 0.53888 | 0.00016049 | 0.546108 |
| PROPENE | 0 | 0.026482 | 0 | 0 | 0.248889 | 0.290356 | 0.00018567 | 0.294251 |
| WATER | 0 | 0 | 0 | 1 | 0.152151 | 0.011367 | 0.9965196 | 0.011519 |
| ETHENE | 0 | 0.019232 | 0 | 0 | 0.018074 | 0.021086 | 7.0981E−06 | 0.021369 |
| ETHANE | 0 | 0.036695 | 0 | 0 | 0.034912 | 0.040233 | 0.00299625 | 0.040773 |
| CO2 | 0 | 0.000027 | 0 | 0 | 0.01137 | 0.013266 | 0 | 0.00003 |
| METHANE | 0 | 0 | 0 | 0 | 0 | 0 | 0 | 0 |
| H2 | 0 | 0.02996 | 0 | 0 | 0.028155 | 0.03285 | 0 | 0.03329 |
| O2 | 0 | 0 | 0.99 | 0 | 0 | 0 | 0 | 0 |
| N2 | 0 | 0.00885 | 0.01 | 0 | 0.008317 | 0.009703 | 0 | 0.009833 |
| CO | 0 | 0.038426 | 0 | 0 | 0.036111 | 0.042132 | 0 | 0.042697 |
| Temperature | 18.36 | 450 | 600 | 350 | 600 | 55 | 55 | 55 |
| Pressure [bar] | 8 | 8 | 2.5 | 40 | 2.5 | 10 | 2.5 | 10 |

| Stream No. | 9 | 10 | 11 | 12 | 13 | 14 | 15 |
|---|---|---|---|---|---|---|---|
| Amount [kg/h] | 2211.53494 | 162968.709 | 1897.53461 | 43749.9998 | 119218.708 | 11921.8708 | 107296.837 |
| BUTANE | 0 | 0.00013 | 0 | 0 | 0.000177 | 0.000177 | 0.000177 |
| PROPANE | 0 | 0.552467 | 0 | 0.002 | 0.754473 | 0.754473 | 0.754473 |
| PROPENE | 0 | 0.297677 | 0 | 0.997963 | 0.040692 | 0.040692 | 0.040692 |
| WATER | 0 | 0.00001 | 1 | 0.000037 | 0 | 0 | 0 |
| ETHENE | 0 | 0.021618 | 0 | 0 | 0.029551 | 0.029551 | 0.029551 |
| ETHANE | 0 | 0.041248 | 0 | 0 | 0.056385 | 0.056385 | 0.056385 |
| CO2 | 1 | 0.00003 | 0 | 0 | 0.000041 | 0.000041 | 0.000041 |
| METHANE | 0 | 0 | 0 | 0 | 0 | 0 | 0 |
| H2 | 0 | 0.033678 | 0 | 0 | 0.046037 | 0.046037 | 0.046037 |
| O2 | 0 | 0 | 0 | 0 | 0 | 0 | 0 |
| N2 | 0 | 0.009948 | 0 | 0 | 0.013598 | 0.013598 | 0.013598 |
| CO | 0 | 0.043194 | 0 | 0 | 0.059045 | 0.059045 | 0.059045 |
| Temperature | 55 | 60 | 30 | 50 | 100 | 100 | 100 |
| Pressure [bar] | 10 | 10 | 10 | 1 | 10 | 8 | 8 |

1-15. (canceled)

16. A process for preparing propene from propane, comprising:
A) providing a feed gas stream (a) comprising propane;
B) feeding said feed gas stream (a) comprising propane and, optionally, steam and an oxygenous gas stream into a dehydrogenation zone and dehydrogenating said propane to propene to obtain a product gas stream (b) comprising propane, propene, methane, ethane, ethene, hydrogen, carbon monoxide, carbon dioxide, steam, and oxygen;
C) cooling said product gas stream (b) and, optionally, compressing and removing steam by condensation from said product gas stream (b) to obtain a steam-depleted product gas stream (c);
D) contacting said product gas stream (c) with a selective adsorbent which adsorbs propene selectively under selected adsorption conditions to obtain a propene-laden adsorbent and a propene-depleted gas stream (d2) comprising propane, methane, ethane, ethene, hydrogen, carbon monoxide, and carbon dioxide; and
E) releasing a gas stream (e1) comprising propene from said propane-laden adsorbent by pressure reduction and/or heating of said adsorbent.

17. The process of claim 16, wherein said dehydrogenation in B) is an oxidative or a nonoxidative dehydrogenation.

18. The process of claim 16, wherein said dehydrogenation in B) is carried out adiabatically or isothermally.

19. The process of claim 16, wherein said dehydrogenation in B) is carried out in a fixed bed reactor, a moving bed reactor, or a fluidized bed reactor.

20. The process of claim 16, wherein an oxygenous gas stream is fed into said dehydrogenation zone of B), said oxygenous gas stream comprising at least 90% by volume of oxygen.

21. The process of claim 20, wherein said dehydrogenation in B) is carried out as an autothermal dehydrogenation.

22. The process of claim 16, wherein said selective adsorbent is an adsorbent comprising a porous metal-organic framework material, molecular sieve, activated carbon, silica gel, and/or a porous covalent organic framework material.

23. The process of claim 22, wherein said porous metal-organic framework material comprises at least one metal ion selected from the group consisting of Mg, Al, In, Cu, Zn, Fe, Zr, and Y.

24. The process of claim 22, wherein said molecular sieve is selected from the group consisting of 4A molecular sieves, 5A molecular sieves, and 13X molecular sieves.

25. The process of claim 16, wherein said propane-comprising offgas stream (d2) is at least partly recycled into said dehydrogenation zone.

26. The process of claim 16, wherein, before prior to D), carbon dioxide is removed from said product gas stream (c) by gas scrubbing or by absorption on solid absorbents.

27. The process of claim 16, further comprising
F) contacting at least a portion of said propane-comprising gas stream (d2) with a high-boiling absorbent and subsequently desorbing the gases dissolved in said high-boiling absorbent to obtain a recycle stream (f1) consisting essentially of propane and an offgas stream (f2) comprising methane, ethane, ethene, hydrogen, carbon monoxide, and carbon dioxide, and recycling said recycle stream (f1) into said dehydrogenation zone.

28. The process of claim 27, said desorption is achieved by stripping with steam.

29. The process of claim 27, wherein an adsorptive workup is performed as an alternative to or in addition to the absorption in F).

30. The process of claim 16, further comprising
F) removing carbon dioxide by gas scrubbing from at least a substream of said propane-comprising gas stream (d2) to obtain a low-carbon dioxide recycle stream (f1), which is recycled into said dehydrogenation zone.
